(19) United States
(12) Patent Application Publication
(10) Pub. No.: US 2025/0335875 A1
(43) Pub. Date: Oct. 30, 2025

(54) SYSTEM AND METHOD FOR ACCOMMODATION ASSESSMENT

(71) Applicant: MAGNIFY ACCESS INC., North York (CA)

(72) Inventors: Keri BANKA, North York (CA); Lyle WILLIAMS, North York (CA)

(73) Assignee: MAGNIFY ACCESS INC., North York (CA)

(21) Appl. No.: 19/189,484

(22) Filed: Apr. 25, 2025

Related U.S. Application Data
(60) Provisional application No. 63/639,355, filed on Apr. 26, 2024.

(51) Int. Cl. G06Q 10/105; G06F 40/40; G16H 10/60; G16H 50/30

(52) U.S. Cl. CPC G06Q 10/105; G06F 40/40; G16H 10/60; G16H 50/30

(57) ABSTRACT

A system and method are described for providing an accommodation assessment including a recommended accommodation based on personalized (dynamic) data and common (static) data. In a first step, an input comprising a combination of both personalized and common data is entered. In a second step, the system associates the individualized data with the common data. The association is undertaken with the help of a trained AI model that better understands the relationship between different types of disability-related data and potential workplace accommodations, and therefore identifies patterns, correlations, and/or insights that can inform the accommodation. The AI model is then further utilized to identify accommodation(s) and generate an accommodation assessment for the user. The system can receive a desired data accuracy threshold, and if the system determines that the threshold has not been met, further information may be requested, or the data is sent to an employer for further review.

### CROSS REFERENCE TO RELATED APPLICATION

The present application claims priority to U.S. Provisional Application No. 63/639,355, entitled “SYSTEM AND METHOD FOR ACCOMMODATION ASSESSMENT” filed on Apr. 26, 2024, the contents of which are incorporated herein by reference in their entirety.

### FIELD

The invention relates generally to accommodation systems and more particularly, to a system and method for accommodation assessment.

### BACKGROUND

The proposed disclosure stems from the recognition of challenges encountered in the field of workplace accommodations, especially regarding individuals with disabilities. Historically, the process of identifying, implementing, and evaluating workplace accommodations has been fraught with inefficiencies, inconsistencies, and barriers that hinder both employers and employees. Accommodations for people with disabilities are, in most cases, unique to the individual requesting them. Practitioners use a combination of the employee narrative, medical documentation, and supplementary questionnaires to formulate accommodation plans. The challenge to this process is that the awareness of accommodations is limited by the practitioners' knowledge and the information presented to them.

One of the consequences of this human-based approach is that it introduces exorbitant delays in reviewing and determining appropriate workplace accommodations. As well as placing tremendous emotional labour on the employees with disabilities to retell their challenges and discomforts to essentially a stranger, often in a position of power. This practice can result in judgment, prejudice, and discrimination in the workplace. Additionally, identifying workplace accommodation requires a level of human judgment in evaluating the necessity of one accommodation over the other. This decision-making process is fraught with potential bias and discrimination on behalf of the practitioner, whether consciously or subconsciously.

As such there is a need for a solution that alleviates the aforementioned challenges in providing workplace accommodations. Indeed, there is a need for an autonomous approach that seeks information from the employee and proposes appropriate and supportive workplace accommodations. Preferably, employees simply upload any documentation and complete an adaptive questionnaire to gather an understanding of the impact of their disability in relation to their workplace environment.

There is a need for a system that leverages a large language model (LLM) and associates functional limitations with environments and workplace accommodations. Preferably, a LLM would use a large, trained dataset to determine appropriate and supportive accommodations for persons with disabilities. The process could take minutes instead of several days across multiple meetings.

### SUMMARY

In an aspect, the present disclosure provides a computer-implemented method of providing an accommodation assessment, the steps comprising: receiving an input, the input comprising at least individualized user data, the individualized user data related to at least one disability of a user; utilizing an artificial intelligence (AI) model to associate the individualized user data with common data, the common data related to at least one of: workplace environment, workplace accommodation and functional impact; generating the accommodation assessment based on the association generated by the AI; determining whether the accommodation assessment is within a score threshold; and, based on the determination, finalizing and sending the accommodation assessment.

In an aspect, the present disclosure provides a non-transitory computer-readable media storing computer-readable instructions that when executed by at least one processor cause the at least one processor to perform operations, the operations comprising: receiving an input, the input comprising at least individualized user data, the individualized user data related to at least one disability of a user; utilizing an artificial intelligence (AI) model to associate the individualized user data with common data, the common data related to at least one of: workplace environment, workplace accommodation and functional impact; generating the accommodation assessment based on the association generated by the AI; determining whether the accommodation assessment is within a score threshold; and, based on the determination, finalizing and sending the accommodation assessment.

### BRIEF DESCRIPTION OF THE DRAWINGS

A detailed description of embodiments of the invention is provided herein below, by way of example only, with reference to the accompanying drawings, in which:

FIG. 1 is a flowchart of a system and method for accommodation assessment, according to an embodiment of the present disclosure; and,

FIG. 2 is a flowchart of a system and method for accommodation assessment, according to another embodiment of the present disclosure.

It is to be expressly understood that the description and drawings are only for the purpose of illustration of certain embodiments of the invention and are an aid for understanding. They are not intended to be a definition of the limits of the invention.

### DETAILED DESCRIPTION

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

It is understood that the presently described method can be performed by a processor in a device. In an embodiment, the processor may be part of a system that would perform various aspects of the techniques described in this disclosure. The system would be comprised of a user device (not shown) capable of running the method, the user device in wireless communication with a host device server and other external devices to perform the various functions. The host device may be any type of computing device capable of running the functions as described in the present disclosure. The host device may include one or more servers, execution platforms, ML/AI units and databases.

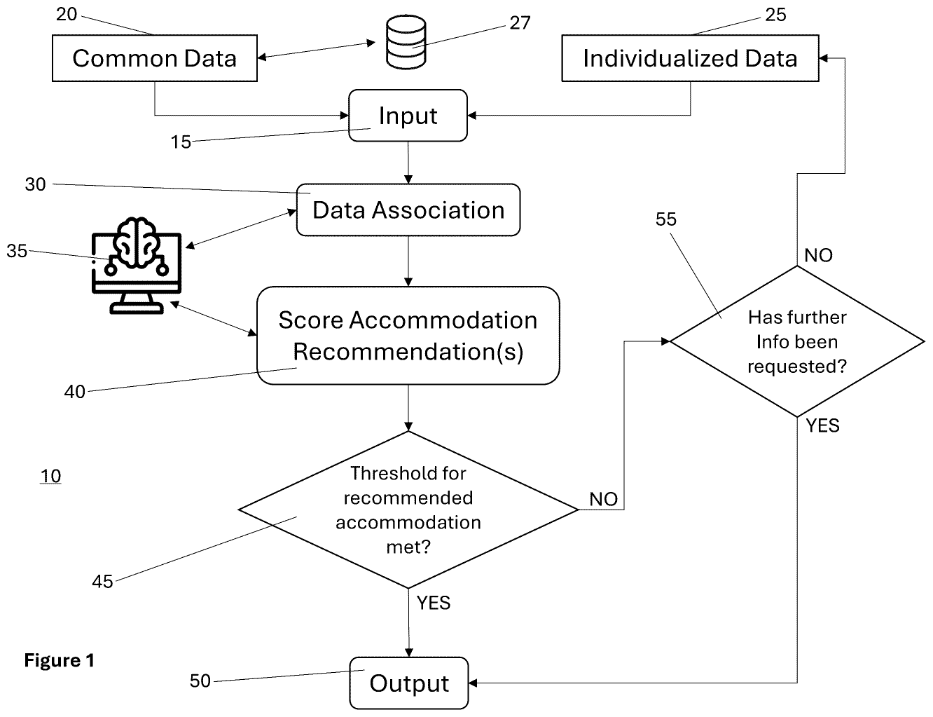

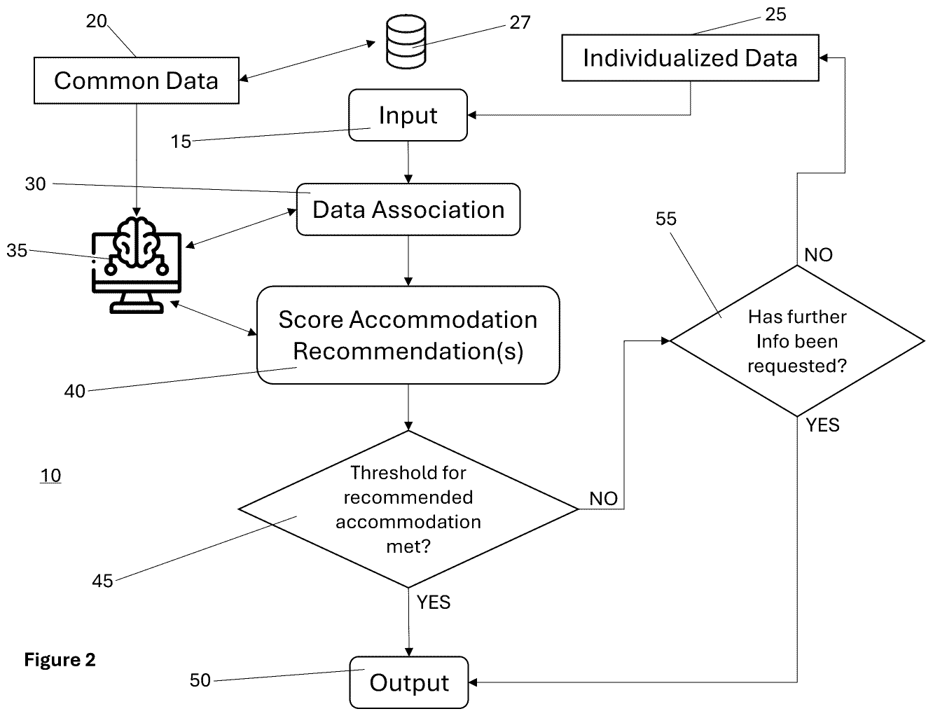

With reference to FIG. 1 and according to an embodiment of the present disclosure, a method for providing an accommodation assessment 10 is shown, the method 10 being performed within a system. In a first step 15, an input is provided, the input consisting of both common data 20 and individualized data 25. It is understood that the common data 20 is stored within a database 27. Common data 20 is defined as a dataset of known information, including but not limited to: workplace environments (e.g. office, factory), workplace accommodations (e.g. ergonomic chair, text-to-speech), functional impact of the disability (e.g. sensitivity to light, chronic wrist pain) and types of disabilities. The common data 20 also includes questions to be used by the system to build the below-referenced questionnaire for the user. This common data 20 dataset may be updated regularly so that it contains the most recent information available and can make the output as reliable as possible. For individualized data 25, an individual is prompted with a questionnaire or another input form to understand the user's disability or impairment. The user may provide a written narrative (raw text input) of the impact of the disability and the user also has the opportunity to input documentation that identifies the functional impacts, diagnoses, accommodations. Documentation that can be uploaded includes, but is not limited to, medical notes or reports, psychoeducational assessments, etc. Once the various individualized data 25 has been entered, a second step 30 of the method 10 is to combine and associate the common data 20 with the individualized data 25 by using an artificial intelligence (AI) model 35. The data association block performs a variety of tasks, including data preprocessing and mapping. For preprocessing, the system prepares the common and individual data 20, 25, by formatting and labelling it for AI processing. For example, the system may include tasks such as separating paragraphs into sentences, removing titles, or anonymizing personal information. Once the data has been preprocessed, it is sent to the AI model 35 for analysis. In this embodiment, the AI model 35 is trained and configured to take the individual data 25 (which is dynamic by nature) and associate it with common data 20 (which is static by nature). In an embodiment, the system is configured to receive both the individual dynamic data 25 and the common static data 20 at the input step 15. However, this may be computationally intensive, resulting in a more complex and time-consuming implementation of the system. Therefore, in another embodiment, the system is configured such that the AI model 35 is pre-trained (or trained less frequently) using the common static data 20, such that only the individual dynamic data 25 is used at the input step 15. Such an alternate embodiment is illustrated in FIG. 2. The AI model 35 is trained to understand the relationship between different types of disability-related data and potential workplace accommodations. The AI model 35 is configured to take into consideration the disability, the impact of the disability on the individual and the impact of the work environment. In doing so, the model 35 is better able to determine appropriate workplace accommodations that suit the specific environment. In other words and by way of example not intended to be limiting, the model 35 may conclude that a person with a hearing-based disability who works on a loud factory floor would not benefit from hearing aids exclusively and would benefit from visual alerts or haptic devices to notify them. The AI model 35 processes and analyzes the combined common and individual data 20, 25 to identify patterns, correlations, and insights that can inform the accommodation. Once the individualized and common data 25, 20 are associated, the system uses insights generated by the AI model 35 to identify the accommodation. The accommodation recommendations consider factors such as the nature of the disability, the functional limitations it presents, and the potential workplace accommodations that could address those limitations. The analysis, and more specifically the workplace accommodations that are recommended, are scored in a third step 40. More particularly, the system searches for similarities between prediction and truth, based on a variety of factors, including word and phrase overlap, and such an analysis is scored in a third step 40. In this embodiment, the user can choose the type of scoring to be used and preferred threshold to achieve. In this embodiment, a BLEU (bilingual evaluation understudy) algorithm can be used to score the analysis. The BLEU algorithm generates a score between 0 and 1 to evaluate the quality of the prediction in comparison to its knowledge database. In this way, the system permits the user to define the type of threshold it wants and evaluate the scoring achieved. Indeed, in a fourth step 45, the system determines whether the recommendation accommodations meet the predetermined threshold. Although in this embodiment, the threshold is a BLEU score of 0.8, other types of thresholds or associated values may be possible. If the requisite score is achieved, the system outputs the recommendations in a fifth step 50. Once the recommendations have been provided, the user is able to review the recommendations and select the any one or more they wish to send to their employer. Therefore, in an optional subsequent step (not shown), the system sends the selected recommendations to the employer for consideration. Conversely, if the requisite score is not achieved, the system then determines in an optional sixth step 55 whether more information has been requested or not, and if so, whether a certain limit has been met. Indeed, it is an object of the disclosure that if the score threshold cannot be achieved, it is desirable to obtain more individualized data 25 and restart the process, as sometimes additional information may further help the system in determining the recommended accommodation more accurately. If further information has already been requested, or if it has been requested the predetermined number of times, the system returns to the fifth step 50 and outputs the recommended accommodation, even if below the desired threshold. This allows an evaluator to nevertheless review the output and apply additional subjective judgment regarding a possible outcome.

Although various embodiments of the present invention have been described and illustrated, it will be apparent to those skilled in the art that numerous modifications and variations can be made without departing from the scope of the invention, which is defined in the appended claims.

1. A computer-implemented method of providing an accommodation assessment, the steps comprising:
receiving an input, the input comprising at least individualized user data, the individualized user data related to at least one disability of a user;
utilizing an artificial intelligence (AI) model to associate the individualized user data with common data, the common data related to at least one of: workplace environment, workplace accommodation and functional impact;
generating the accommodation assessment based on the association generated by the AI;
determining whether the accommodation assessment is within a score threshold; and,
based on the determination, finalizing and sending the accommodation assessment.

2. The method of providing an accommodation assessment of claim 1 wherein the AI model is configured to query the user to finetune the accommodation assessment.

3. The method of providing an accommodation assessment of claim 1 wherein the individualized user data is comprised of at least one of: a user narrative and user medical documentation.

4. The method of providing an accommodation assessment of claim 1 further comprising the step of: sending the generated accommodation assessment to an employer of the user for consideration.

5. The method of providing an accommodation assessment of claim 1 further comprising the step of: if the accommodation assessment falls outside the score threshold, requesting additional information from the user.

6. The method of providing an accommodation assessment of claim 5, further comprising the steps of:
if the additional information results in another accommodation assessment that falls outside the score threshold, issuing a notice; and,
if the additional results in a second accommodation assessment that falls within the score threshold, sending the second accommodation assessment to a third party.

7. The method of providing an accommodation assessment of claim 1, wherein the input is comprised of the common data.

8. The method of providing an accommodation assessment of claim 1, wherein the AI model is trained to take into consideration the disability, the impact of the disability on the user and an impact to the workplace environment.

9. The method of providing an accommodation assessment of claim 1, further comprising the step of utilizing an algorithm to score the accommodation assessment.

10. The method of providing an accommodation assessment of claim 9, wherein the algorithm is a bilingual evaluation understudy (BLEU).

11. The method of providing an accommodation assessment of claim 10, wherein the BLEU score threshold is 0.8.

12. A data processing system comprising at least one processor for carrying out the steps of the method of claim 1.

13. A non-transitory computer-readable media storing computer-readable instructions that when executed by at least one processor cause the at least one processor to perform operations, the operations comprising:
receiving an input, the input comprising at least individualized user data, the individualized user data related to at least one disability of a user;
utilizing an artificial intelligence (AI) model to associate the individualized user data with common data, the common data related to at least one of: workplace environment, workplace accommodation and functional impact;
generating the accommodation assessment based on the association generated by the AI;
determining whether the accommodation assessment is within a score threshold; and,
based on the determination, finalizing and sending the accommodation assessment.

14. The non-transitory computer-readable media storing computer-readable instructions of claim 13 wherein the AI model is configured to query the user to finetune the accommodation assessment.

15. The non-transitory computer-readable media storing computer-readable instructions of claim 13 wherein the individualized user data is comprised of at least one of: a user narrative and user medical documentation.

16. The non-transitory computer-readable media storing computer-readable instructions of claim 13 further comprising the step of: sending the generated accommodation assessment to an employer of the user for consideration.

17. The non-transitory computer-readable media storing computer-readable instructions of claim 13 further comprising the step of: if the accommodation assessment falls outside the score threshold, requesting additional information from the user.

18. The non-transitory computer-readable media storing computer-readable instructions of claim 17, further comprising the steps of:
if the additional information results in another accommodation assessment that falls outside the score threshold, issuing a notice; and,
if the additional results in a second accommodation assessment that falls within the score threshold, sending the second accommodation assessment to a third party.

19. The non-transitory computer-readable media storing computer-readable instructions of claim 13, wherein the input is comprised of the common data.

20. The non-transitory computer-readable media storing computer-readable instructions of claim 13, wherein the AI model is trained to take into consideration the disability, the impact of the disability on the user and an impact to the workplace environment.
